(19) United States
(12) Patent Application Publication
(10) Pub. No.: US 2024/0366495 A1
(43) Pub. Date: Nov. 7, 2024

(54) COMPOSITION FOR AMELIORATING WRINKLES USING FERMENTED EXTRACT OF CANNABIS STEM AND PREPARATION METHOD THEREOF

(71) Applicant: KOREA INSTITUTE OF SCIENCE AND TECHNOLOGY, Seoul (KR)

(72) Inventors: Young Tae PARK, Gangneung-si (KR); Jisu PARK, Gangneung-si (KR); Huitae MIN, Gangneung-si (KR); Taehyun KIM, Gangneung-si (KR); Jin-Woo KIM, Gangneung-si (KR); Tae Jung KIM, Gangneung-si (KR); Jin Chul KIM, Gangneung-si (KR); Jung Yeob HAM, Gangneung-si (KR)

(73) Assignee: KOREA INSTITUTE OF SCIENCE AND TECHNOLOGY, Seoul (KR)

(21) Appl. No.: 18/404,121

(22) Filed: Jan. 4, 2024

(30) Foreign Application Priority Data
May 2, 2023 (KR) 10-2023-0057008

(51) Int. Cl. A61K 8/9789; A61K 36/185; A61Q 19/08

(52) U.S. Cl. CPC A61K 8/9789; A61K 36/185; A61Q 19/08; A61K 2236/15; A61K 2236/19; A61K 2236/333; A61K 2800/782; A61K 2800/85

(57) ABSTRACT

The present invention relates to a use of a fermentation product of Lacticaseibacillus casei of cannabis stems and an extract thereof for ameliorating skin wrinkles caused by photoaging, and to an anti-wrinkle cosmetic composition or pharmaceutical composition including the fermentation product.

### CROSS-REFERENCE TO RELATED APPLICATION

This application claims priority to and the benefit of Korean Patent Application No. 10-2023-0057008, filed May 2, 2023, the disclosure of which is incorporated herein by reference in its entirety.

### BACKGROUND

### 1. Field of the Invention

The present invention relates to a cosmetic composition for ameliorating wrinkles using a microbial fermentation product of a cannabis stem extract.

### 2. Discussion of Related Art

Cannabis (Cannabis sp.) is an annual plant that belongs to the Cannabaceae family, is native to Central Asia, grows well in a mild and humid climate, and is known to have been cultivated in Korea since the 1st century BC. The fibers of cannabis are usually used to make fabric or thread, and hemp seeds, with their skins removed, are used as a raw material for health foods and oils. Hemp seeds are known to have various activities such as antioxidant actions, strengthening of immunity, prevention of arteriosclerosis, amelioration of hypertension, improving hyperlipidemia, anti-inflammatory and anti-cancer actions, and in traditional Chinese medicine prescription books such as Dongui Bogam, it is recorded that cannabis was used as a therapeutic agent for various diseases.

However, particularly in Korea, the Narcotics Control Act stipulates that cannabis parts excluding stems and roots, cannabis resin, and cannabinoid components THC, CBD, and CBN are narcotic ingredients, so that there are limitations to the development of therapeutic agents and the commercialization of functional products using CBD. Therefore, attempts have been made to develop by-products such as stems and roots, other than raw materials strictly regulated by law such as cannabis leaves and unfertilized female flowers, into functional materials after narcotic components are removed and processed.

Repeated exposure to ultraviolet light results in increased collagenase and denaturation and destruction of collagen fibers, thereby causing skin damage such as reduction of skin elasticity and promotion of wrinkle formation. In skin exposed to ultraviolet light, fibroblast division and decrease in fibroblast number occur, resulting in a decrease in overall collagen due to a decrease in collagen synthesis and an increased production of collagenase, thereby leading to the formation of wrinkles. Excessive UV light accelerates photoaging, which causes pigmentation, roughness, loss of elasticity and wrinkles to the skin, and may induce diseases including skin cancer in severe cases. Further, excessive UV light induces reactive oxygen species in skin cells, which induce the deformation and loss of function of cell components. Moreover, excessive UV light induces the production of collagenase to degrade collagen and elastin, resulting in wrinkles.

Compared to intrinsic aging, which occurs over time, structural and physiological changes in extrinsic aging are more evident to the naked eye. Therefore, there is a need for the development of a cosmetic for effectively ameliorating photoaging.

### SUMMARY OF THE INVENTION

Accordingly, the present inventors have conducted research to develop a skin functional material using cannabis stems, and found that when the cannabis stem raw material or extract was fermented through lactic acid bacteria, wrinkles could be alleviated, particularly the effect of ameliorating wrinkles caused by photoaging was excellent, thereby completing the present invention.

Therefore, an object of the present invention is to provide a composition for ameliorating skin wrinkles, including a Lacticaseibacillus casei (KCTC 3109) fermentation product of cannabis stems as an active ingredient.

To achieve the aforementioned object, the present invention provides a composition for ameliorating skin wrinkles, including a Lacticaseibacillus casei (KCTC 3109) fermentation product of cannabis stems as an active ingredient. Hereinafter, the present invention will be described in detail.

An aspect of the present invention, the present invention relates to a composition for ameliorating skin wrinkles, including a Lacticaseibacillus casei (KCTC 3109) fermentation product of a cannabis stem extract.

In this specification, cannabis (Cannabis sp.) is an annual plant of the Cannabaceae family, and is referred to as “cannabis” or “hemp.” In the present invention, the underground parts (roots) and stem parts of the cannabis plant excluding leaves and seeds, were separated and used. Cannabis stems may be used either by crushing a raw material or as an extract using a solvent.

In the case of extracts, the extraction method is not limited, and extraction may be performed using any known extraction method or solvent. For example, the extract may be extracted using a hot water extraction method, a cold immersion method, an ultrasonic extraction method, a filtration method, a reflux extraction method, or a combination of two or more of these extraction methods, and is not limited to these methods.

Further, the type of solvent used to prepare the extract is not particularly limited, and any known solvent may be used. Although not limited thereto, water; a lower alcohol such as ethanol, methanol, propyl alcohol, and butyl alcohol; a polyhydric alcohol such as glycerin, butylene glycol, and propylene glycol; and a hydrocarbon solvent such as ethyl acetate, methyl acetate, acetone, benzene, hexane, diethyl ether, and dichloromethane or a mixture of the solvents may be used as the solvent, and preferably, the extract may be extracted with ethanol alone or a mixed solvent of ethanol and water.

The extract may include an extract obtained by subjecting cannabis stems to extraction treatment, a diluted solution or concentrated solution of the extract, a dried product of the extract, a crude extract or purified product of the extract, or a mixture thereof, and includes the extract itself or all formulations which may be obtained using the same.

In addition, the extract of the present invention may be a fermented product fermented using microorganisms. As the microorganism, lactic acid bacteria may be used, and preferably a microorganism of the genus Lacticaseibacillus or Paenibacillus may be used, and more preferably, a Lacticaseibacillus casei (KCTC 3109) strain may be used. Through experiments, it was confirmed that when cannabis stems are fermented using Lacticaseibacillus casei (KCTC3109), cytotoxicity is alleviated and the anti-wrinkle effect against UV light is much greater than that of other lactic acid bacteria. The fermented product may be used in the form of a solution or dried product, and is not limited in its form. Furthermore, the aforementioned fermented product may include a raw material fermentation product or a fermented product of a crude extract of cannabis stems.

The composition of the present invention has an effect of ameliorating wrinkles, and the effect may be an amelioration of skin wrinkles caused by photoaging. Photoaging is extrinsic aging during skin aging, and refers to the aging caused by chronic exposure to sunlight, unlike intrinsic aging. Photoaging refers to aging caused by sunlight, and may preferably correspond to aging caused by ultraviolet (UV) light among sunlight.

As used herein, ‘active ingredient’ refers to an ingredient capable of exhibiting a desired activity alone or exhibiting the activity together with a carrier that is inactive per se. The composition for ameliorating wrinkles of the present invention includes, as an active ingredient, a fermented product of the raw material or crude extract of a Lacticaseibacillus casei (KCTC3109) strain of a cannabis stem extract, and may include various other additives.

The composition of the present invention may be utilized as a cosmetic, a pharmaceutical composition, or a food composition depending on the purpose.

As an aspect of the present invention, the composition of the present invention may be a cosmetic composition. The “cosmetic” is a product that is used in similar ways, such as by applying, rubbing, or spraying on the human body in order to maintain or improve the health of the skin or hair and has a mild effect on the human body, and particularly, the cosmetic of the present invention refers to a functional cosmetic, which has specialized functionality, in which physiological efficacy and effects are emphasized unlike general cosmetics, and specific efficacy and effects such as amelioration of wrinkles are emphasized, as stipulated by the Cosmetic Act.

The ‘wrinkle improving cosmetic’ of the present invention may contain the fermented cannabis stem extract of the present invention in an amount of 0.001 to 30 parts by weight based on 100 parts by weight of the entire cosmetic composition, but is not limited to this content, and may include the fermented extract by arbitrarily adjusting a content range capable of exhibiting efficacy depending on the formulation of the cosmetic.

The cosmetic composition of the present invention may additionally include one or more of cosmetic raw materials registered and notified by the Ministry of Food and Drug Safety and raw materials registered with ICID. That is, it is possible to include one or more of an organic solvent, a solubilizer, a thickener, a gelling agent, a softener, an antioxidant, a suspending agent, a stabilizer, a flavoring agent, a surfactant, an emulsifier, a filler, a sequestrant, a preservative, a vitamin, a blocking agent, a wetting agent, a dye and pigment, a hydrophilic or lipophilic active agent, or an auxiliary typically used in the cosmetic field such as any other components typically used in cosmetics.

The cosmetic composition of the present invention can be prepared in any formulation typically prepared in the art to which the present invention pertains. The cosmetic composition of the present invention may have, for example, a formulation such as a softening lotion, an astringent lotion, a nutritional lotion, a nutritional cream, a massage cream, an eye cream, an eye essence, an essence, a cleansing cream, a cleansing emulsion, a cleansing foam, cleansing water, a pack, a powder, a body lotion, a body cream, a body essence, a body cleanser, a hair dye, a shampoo, a conditioner, a hair styling agent, a hair restorer, an ointment, a gel, a cream, a patch, an aerosol, a powder and a skin adhesive type, but the formation is not limited thereto. Further, in each formulation, other components in addition to the aforementioned essential components may be suitably selected and blended by a person with ordinary skill in the art without difficulty depending on the type of external preparation or the purpose of use, and the like.

The cosmetic compositions may be provided in any formulation suitable for topical application. For example, the cosmetic composition may be provided in the form of a solution, an emulsion obtained by dispersing an oil phase in an aqueous phase, an emulsion obtained by dispersing an aqueous phase in an oil phase, a suspension, a solid, a gel, a powder, a paste, a microneedle, a foam, or an aerosol composition. Compositions in such formulations may be prepared by typical methods in the related art.

In addition, the cosmetic composition according to the present specification may further include a moisturizing agent, an emollient, a surfactant, an ultraviolet absorber, a preservative, a bactericide, an antioxidant, a pH adjuster, organic and inorganic pigments, a fragrance, a cooling agent, or an antiperspirant. The blending amounts of the above components can be easily selected by the person with ordinary skill in the art within a range that does not impair the purpose and effects of the present specification.

As another aspect of the present invention, the composition of the present invention may be a pharmaceutical composition. The ‘pharmaceutical composition’ is a pharmaceutical and may have a preventive or therapeutic effect on a skin disease, specifically a skin disease caused by photoaging, and the disease may include pigmentation, melasma, keratosis, skin cancer, and the like.

The pharmaceutical composition may be administered orally, parenterally, rectally, topically, transdermally, intravenously, intramuscularly, intraperitoneally, subcutaneously, and the like. Dosage forms for oral administration may be tablets, pills, soft and hard capsules, granules, powders, fine granules, solutions, emulsions or pellets, but are not limited thereto. Dosage forms for parenteral administration may be solutions, suspensions, emulsions, gels, injections, drops, suppositories, patches or sprays, but are not limited thereto.

Furthermore, the dosage forms may be easily prepared by typical methods in the art, and may further include surfactants, excipients, wetting agents, emulsifying accelerators, suspensions, salts or buffers for controlling osmotic pressure, colorants, spices, stabilizers, antiseptics, preservatives, or other commonly used adjuvants.

In addition, the composition of the present invention may be a food composition, particularly, a health functional food, and in particular, the “health functional food” refers to a food manufactured and processed using raw materials or ingredients that have functional properties that are useful for the human body according to Health Functional Food Act No. 6727, and “functional” means intake for the purpose of regulating nutrients for the structure and function of the human body, or obtaining effects useful for health purposes such as physiological effects.

The food or health functional food of the present invention can be manufactured and processed into a pharmaceutical administration form such as a powder, a granule, a pill, a capsule, a pill, a suspension, an emulsion, and a syrup, or as a health functional food such as a tea bag, an infusion, a beverage, a candy, a jelly, and a gum for the purpose of ameliorating skin wrinkles.

The food or health functional food composition of the present invention may be used as a food additive, and may be commercialized alone or in combination with other ingredients. Furthermore, the food or health functional food composition of the present invention may include nutrients, vitamins, electrolytes, flavors, colorants, enhancers, pectic acid and salts thereof, alginic acid and salts thereof, organic acids, protective colloid thickeners, pH adjusters, stabilizers, preservatives, glycerin, alcohols, carbonating agents used in carbonated drinks, and the like. The ingredients may be used alone or in combination, and may be used in combination in suitable amounts.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 confirms the cytotoxicity results of the bacterial strain culture and fermentation product of the present invention;

FIG. 2 shows the results of confirming cytotoxicity of the bacterial strain culture and fermentation product of the present invention and cytotoxicities of vitamin C and ultraviolet (UVB) irradiation as controls;

FIG. 3 shows the results of confirming whether the bacterial strain culture and fermentation product of the present invention changes the cell morphology of dermal fibroblasts;

FIG. 4 shows the results of confirming the MMP-1 expression inhibitory effect of the bacterial strain culture and fermentation product of the present invention for dermal fibroblasts;

FIG. 5 shows the results of confirming the effect of the bacterial strain culture and fermentation product of the present invention on the expression and phosphorylation of wrinkle-forming proteins for dermal fibroblasts; and

FIG. 6 shows the results of confirming and comparing the total phenol content and flavonoid content included in the bacterial strain culture and fermentation product of the present invention.

### DETAILED DESCRIPTION OF EXEMPLARY EMBODIMENTS

Hereinafter, the present specification will be described in detail with reference to Examples in order to specifically explain the present specification. However, the Examples according to the present specification may be modified into various forms, and it should not be interpreted that the scope of the present specification is limited to the Examples described below in detail. The Examples of the present specification are provided to more completely describe the present specification to a person with ordinary skill in the art.

### <Example 1> Preparation of extract, culture solution and fermentation product

A solvent extract of cannabis stems and a lactic acid bacteria fermentation product were prepared. The solvent extract was extracted at room temperature using ethanol or water as a solvent.

The crushed product of cannabis stems and the ethanol extract were added to a medium in which each of Lacticaseibacillus casei, Lacticaseibacillus pentosus, and Paenibacillus barengoltzii was cultured for incubation at 37° C. and 150 rpm for 3 days, thereby producing the raw material fermentation product and crude fermentation product of each microorganism. Thereafter, only the supernatant was separated, extracted with 50% ethanol at room temperature for 1 day, and dried to prepare cultures and fermentation products of the cannabis stem raw material and extract for each strain as shown in the following Table 1.

TABLE 1

| No. | Sample characteristics |
|---|---|
| 1 | Control 1-1 (cannabis stem ethanol extract_distilled water) |
| 2 | Control 1-2 (cannabis stem ethanol extract_10% DMSO) |
| 3 | Control 2-1 (cannabis stem aqueous extract) |
| 4 | L. casei culture solution |
| 5 | L. casei raw material fermentation product |
| 6 | L. casei crude fermentation product |
| 7 | L. pentosus culture solution |
| 8 | L. pentosus raw material fermentation product |
| 9 | L. pentosus crude fermentation product |
| 10 | P. barengoltzii culture solution |
| 11 | P. barengoltzii raw material fermentation product |
| 12 | P. barengoltzii crude fermentation product |

Fermentation was performed by adding a cannabis extract extracted with ethanol.

### <Experimental Example 1> Comparison of Cell Viability

For the culture and fermentation product prepared in Example 1, the cytotoxicity of each sample and the cell viabilities of vitamin C and UVB irradiation as controls were confirmed.

First, the cytotoxicity of each sample was confirmed using the following method. After human dermal fibroblasts (HDFs) were cultured in a 96-well culture plate at a concentration of 1.5×10⁴ cells/well for 24 hours to reach a clustering coefficient of 70 to 80%, and then the sample prepared in Example 1 was treated at a concentration of 0 to 1000 μg/mL, and cultured in an incubator (37° C., 5% CO₂) for an additional 24 hours. After 24 hours, the supernatant was removed from each well, the sample treated with 200 μL of a DMEM culture medium in which MTT had been dissolved at a concentration of 0.5 mg/mL, and incubated for 1 hour, the supernatant was removed again, and then the formazan generated on the culture surface of the culture plate was dissolved in 200 μL of DMSO. The absorbance of each well was measured at 570 nm using a microplate reader.

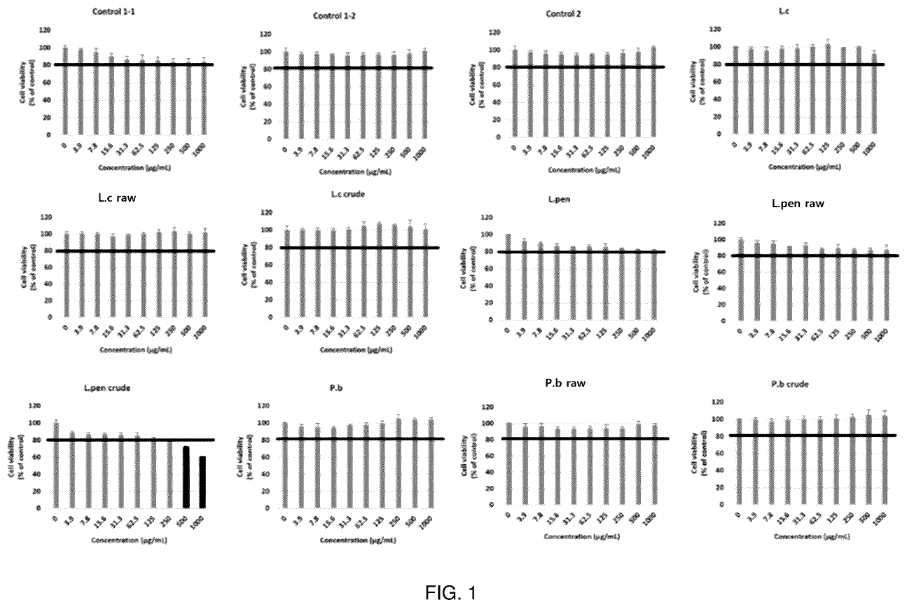

As a result, as illustrated in FIG. 1, it was confirmed that, except for the Lacticaseibacillus pentosus crude extract fermentation product, which is cytotoxic at a concentration of 500 μg/mL, the remaining samples were not toxic until 1000 μg/mL was administered.

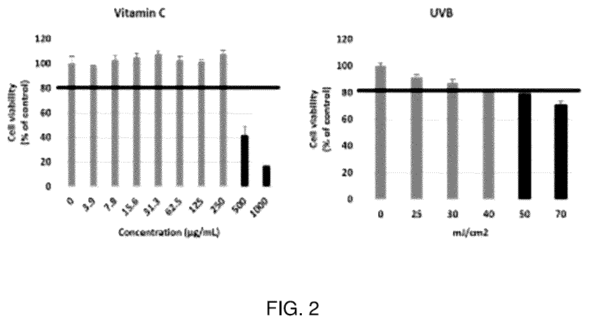

Further, as illustrated in FIG. 2, it was confirmed that when vitamin C was administered, cytotoxicity was exhibited at a concentration of 500 μg/mL or more, and UVB did not have a significant effect on cell viability under treatment conditions of 40 mJ/cm² or less during UVB irradiation.

### <Experimental Example 2> Changes Due to UV Irradiation

### 2-1. Observation of Cell Morphology Changes

An experiment for confirming an anti-wrinkle effect was conducted on each sample confirmed in Example 1. After HDFs were treated with each sample, which did not show cytotoxicity, at the highest concentration, the sample was irradiated with UVB at 25 mJ/cm² after 2 hours and cultured for 24 hours, and then cell photographs were taken and compared.

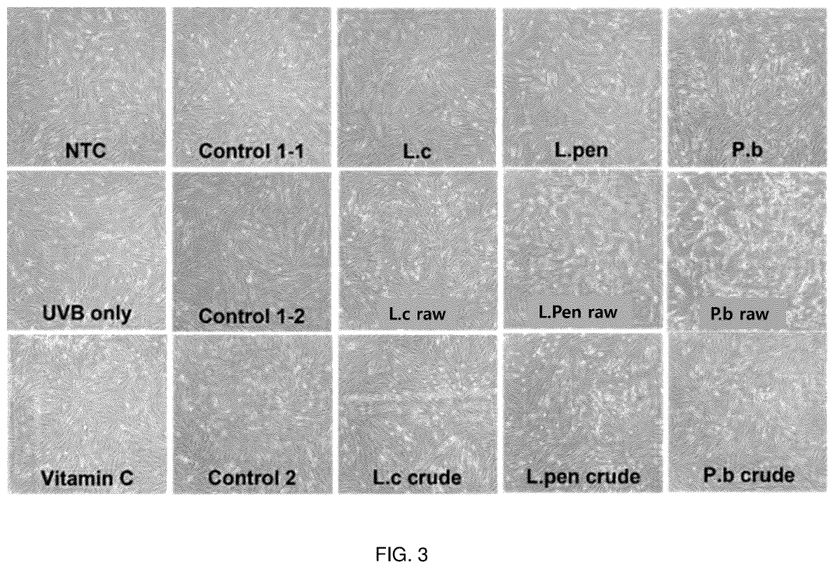

As a result, as illustrated in FIG. 3, it was confirmed that the number of cells decreased in the UVB treatment group compared to the untreated group, and the number of cells increased to the level of the untreated group in the vitamin C treatment group, which is a positive control. In addition, it could be confirmed that, except for the Paenibacillus barengoltzii raw material and fermentation product treatment groups, the number of cells increased to the level of the untreated group. In particular, it could be confirmed that in the case of a group treated with the raw material fermentation product of Lacticaseibacillus casei, cells were present at a higher density than in a group treated with the culture of Lacticaseibacillus casei.

### 2-2. MMP-1 Reduction Effect

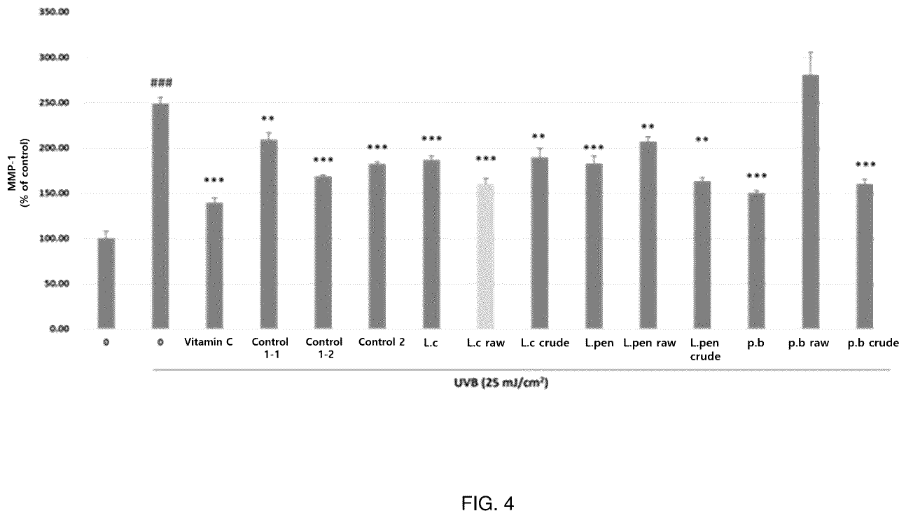

The expression level of MMP-1 was confirmed using ELISA for cells cultured in the same manner as in 2-1 above. As a result, it could be seen that the expression level of MMP-1 was further decreased in the group treated with the raw material fermentation product of Lacticaseibacillus casei compared to the group treated with the culture of Lacticaseibacillus casei (FIG. 4).

### 2-3. Comparison of Changes in Wrinkle-Forming Proteins

For cells cultured in the same manner as in 2-1 above, the effects on the expression levels of p-38, ERK, JNK, p65 (NF-κB), COX2, iNOS, and GAPDH, which are proteins involved in wrinkle formation and the phosphorylation of p-38, ERK, JNK, and p65 (NF-κB) were confirmed by western blot.

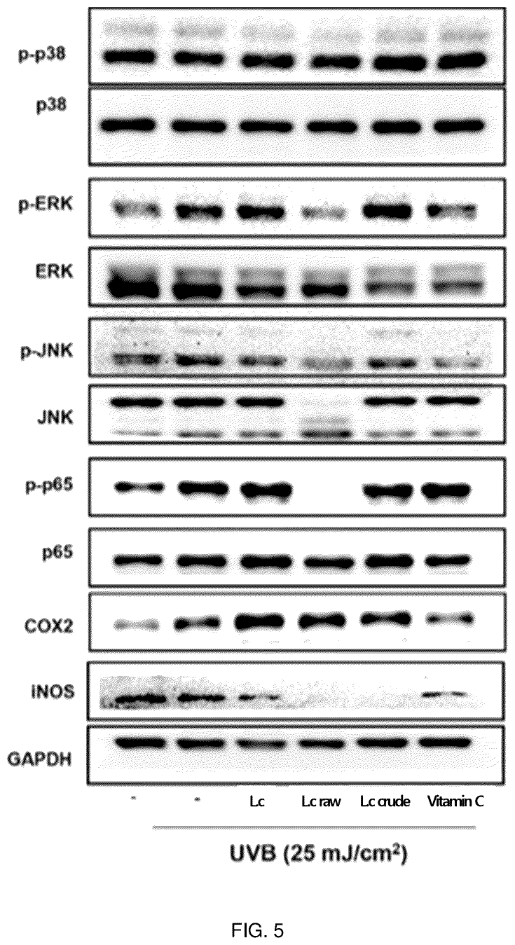

As a result, as illustrated in FIG. 5, it was confirmed that the expression levels and phosphorylation of ERK, JNK, and p65 (NF-κB) increased due to UV irradiation. Furthermore, the expression levels of COX2 and iNOS also increased. However, it was confirmed that when treated with the raw material fermentation product of Lacticaseibacillus casei, the degree of decrease in phosphorylation of ERK, JNK, and p65 (NF-κB) increased, and the expression level of iNOS also decreased.

### <Experimental Example 3> Comparison of Phenol and Flavonoid Contents

The changes in total phenol content and flavonoid content for the raw material fermentation product and culture of Lacticaseibacillus casei were compared.

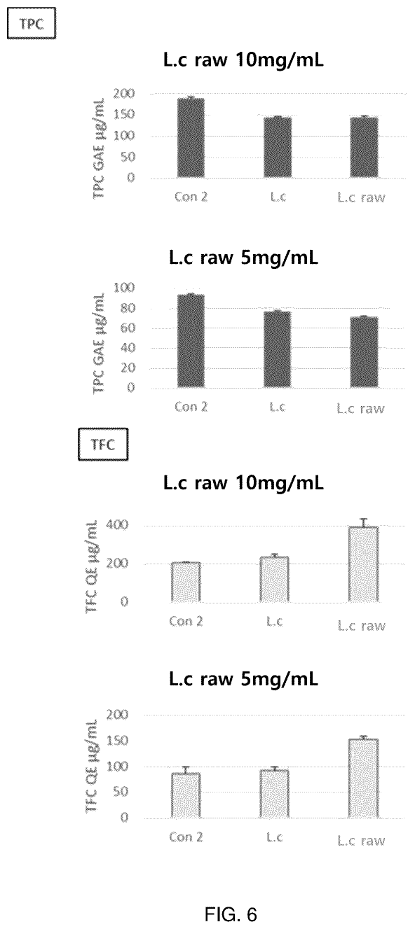

As a result, as illustrated in FIG. 6, it was confirmed that the total phenol content decreased in the raw material fermentation product, but the flavonoid content increased. From these results, it was confirmed that the materials that have the effect of ameliorating wrinkles may be flavonoid-based materials.

The present invention relates to a composition for ameliorating wrinkles, including a Lacticaseibacillus casei strain fermentation product of a cannabis stem extract as an active ingredient, and can be used for a various purposes, such as cosmetics and pharmaceuticals, because an effect of ameliorating wrinkles is enhanced and an effect of reducing cytotoxicity is exhibited.

In the foregoing, the present invention has been examined mainly based on the preferred examples thereof. A person with ordinary skill in the art to which the present invention pertains will be able to understand that the present invention may be implemented in a modified form without departing from the essential characteristics of the present invention. Therefore, the disclosed examples should be considered not from a restrictive viewpoint, but from an explanatory viewpoint. The scope of the present invention is defined not in the above-described description, but in the claims, and it should be interpreted that all differences within a range equivalent thereto are included in the present invention.

What is claimed is:

1. A composition for ameliorating skin wrinkles, comprising a Lacticaseibacillus casei (KCTC 3109) fermentation product of cannabis stems as an active ingredient.

2. The composition of claim 1, wherein the cannabis stem is a crushed raw material or extract.

3. The composition of claim 2, wherein the extract is extracted using an extraction solvent selected from water, ethanol, methanol, propyl alcohol, butyl alcohol, glycerin, butylene glycol, propylene glycol, ethyl acetate, methyl acetate, acetone, benzene, hexane, diethyl ether, dichloromethane and a mixture of these solvents.

4. The composition of claim 3, wherein the composition has MMP-1 production inhibitory activity.

5. The composition of claim 3, wherein the composition has an effect of ameliorating skin wrinkles caused by photoaging.

6. The composition of claim 2, wherein the cannabis stem extract is extracted using a cold immersion method, a hot water extraction method, an ultrasonic extraction method, a filtration method, a reflux extraction method, or a combination of two or more of these extraction methods.

7. The composition of claim 6, wherein the composition has MMP-1 production inhibitory activity.

8. The composition of claim 6, wherein the composition has an effect of ameliorating skin wrinkles caused by photoaging.

9. The composition of claim 2, wherein the composition has MMP-1 production inhibitory activity.

10. The composition of claim 2, wherein the composition has an effect of ameliorating skin wrinkles caused by photoaging.

11. The composition of claim 1, wherein the composition has MMP-1 production inhibitory activity.

12. The composition of claim 1, wherein the composition has an effect of ameliorating skin wrinkles caused by photoaging.

13. The composition of claim 1, wherein the composition is a cosmetic.

14. The composition of claim 1, wherein the composition is a pharmaceutical composition.

15. The composition of claim 1, wherein the composition is a food composition.
